(19) United States
(12) Patent Application Publication
(10) Pub. No.: US 2015/0199487 A1
(43) Pub. Date: Jul. 16, 2015

(54) DEVICE SENSING IN MEDICAL APPLICATIONS

(71) Applicant: KONINKLIJKE PHILIPS N.V., EINDHOVEN (NL)

(72) Inventors: Juris Alex Grauds, Hull, MA (US); Mark David St. Germain, Dunstable, MA (US); Robert Alfred Feuersanger, Westford, MA (US)

(21) Appl. No.: 14/415,430

(22) PCT Filed: Jul. 12, 2013
(86) PCT No.: PCT/IB2013/055746

§ 371 Date: Jan. 16, 2015

Related U.S. Application Data
(60) Provisional application No. 61/673,428, filed on Jul. 19, 2012.
(60) Provisional application No. 61/760,990, filed on Feb. 5, 2013.

(51) Int. Cl. G06F 19/00; A61L 2/10

(52) U.S. Cl. CPC G06F 19/3406; A61L 2/10

(57) ABSTRACT

An accessory enabled device includes a first device (102) including a selectively activated feature (105). An interrogation source (108) is configured to interrogate an accessory device (106), such that, responsive to an interrogation, feedback from the accessory device is measured to determine compatibility between the first device and the accessory device. The selectively activated feature is enabled to perform a task only when compatibility exists between the first device and the accessory device.

### RELATED APPLICATION DATA

This disclosure claims priority to provisional application No. 61/673,428, filed on Jul. 19, 2012 and incorporated herein by reference.

### BACKGROUND

1. Technical Field

This disclosure relates to medical devices and more particularly to sensing device compatibility between devices (e.g., a device and an accessory) to ensure that the devices may properly be employed together to ensure effective and safe treatment of a patient.

2. Description of the Related Art

Infections in patients related to bacterial colonization on skin surfaces are well documented in the medical arts. Infection risk is greatest when medical devices penetrate the skin surface and create an ideal track for bacteria to migrate to sub-dermal tissue and vasculature. This can lead to serious risk of infection. Known methods for achieving disinfection of skin underneath a catheter include using a disinfecting textile, which is inserted underneath the catheter. Use of pulsed ultra-violet (UV) light energy as a way to control skin level bacteria is known; however, the safety and efficacy of the technology remains as an area for improvement. Uncontrolled exposure to UV light can be hazardous to human skin, and can represent an optical hazard from stray light.

Medical devices which interface with disposable accessories are often designed to be sufficiently universal such that the accessories from one brand may be used with capital equipment from a different manufacturer. In some cases, medical devices are designed specifically for use with an accessory of a matching brand, and the efficacy of the device depends upon the use of the system as a whole. It is possible to misuse the device by pairing it with an accessory for which it was not designed, limiting or eliminating the efficacy of the treatment. This can occur due to a plurality of different factors including: operator error (e.g., inadvertent selection of an incorrect accessory), lack of clinical training, unavailability of the appropriate accessories (e.g., due to budget constraints, etc.) and other factors.

Further, the use of medical devices for patient treatment requires the clinician to record the treatment dose and frequency of treatment in the patient's file. This method of record-keeping is vulnerable to human error as the clinician may: forget to record the treatment, record the treatment time or dose incorrectly, or record the data under the wrong patient's record.

### SUMMARY

In accordance with the present principles, an accessory enabled device includes a first device including a selectively activated feature. An interrogation source is configured to interrogate an accessory device, such that, responsive to an interrogation, feedback from the accessory device is measured to determine compatibility between the first device and the accessory device. The selectively activated feature is enabled to perform a task only when a specified compatibility exists between the first device and the accessory device.

A system for determining compatibility between devices includes a first device including a selectively activated feature and a second device including an identification mechanism configured to identify a characteristic of the second device. An interrogation source configured to interrogate the second device, such that, responsive to an interrogation, feedback from the identification mechanism is measured to determine compatibility between the first device and the second device. The selectively activated feature is enabled to perform a task only when compatibility exists between the first device and the second device.

A method for determining compatibility to enable device use includes interrogating an identification mechanism of an accessory device using an interrogation source to generate feedback from the identification mechanism; measuring the feedback to determine compatibility between a first device having a selectively activated feature and the accessory device; and, if the feedback signal indicates compatibility, enabling the selectively activated feature.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a system for determining compatibility between devices in accordance with one embodiment;

FIG. 2 is a perspective schematic view showing a spatial relationship between an interrogation source and an identification mechanism in accordance with one embodiment;

FIG. 3 is a schematic diagram showing a configuration employing RFID interrogation and an RFID tag in accordance with one embodiment;

FIG. 4 is a schematic diagram showing a configuration employing imaging interrogation and an image pattern in accordance with one embodiment;

FIG. 5 is a schematic diagram showing a configuration employing color sensing of a photoluminescent surface in accordance with one embodiment;

FIG. 6 is a schematic diagram showing a configuration employing contact switch interrogation in accordance with one embodiment;

FIG. 7 is a schematic diagram showing a configuration employing inductive interrogation in accordance with one embodiment;

FIG. 8 is a schematic diagram showing a configuration employing visible light interrogation with a visible pattern in accordance with one embodiment; and

FIG. 9 is a flow diagram showing a method for enabling a device in accordance with compatibility between devices in accordance with an illustrative embodiment.

### DETAILED DESCRIPTION OF EMBODIMENTS

In accordance with the present principles, systems and methods for mutual identification of medical devices to determine compatibility are provided. Relationships between devices are determined automatically to decide whether the devices can be properly used together for patient treatment or other uses. In one embodiment, radio-frequency identification (RFID) technology may be employed to permit a medical device to identify an accessory. The medical device may be configured to function only if that particular accessory is present or is determined to be compatible through self-identification. In one example, a dressing sense feature includes a dressing or bandage having an RFID tag therein to be employed with a handheld disinfection device. The disinfection device may include a UV energy source that is enabled in the presence of the RFID tag of the bandage or disposable dressing accessory.

In accordance with another feature, RFID technology may be employed for patient recognition and record keeping as well. This feature may serve as a primary (or secondary) way for tracking which patient received treatment, what time the treatment was delivered, treatment details, etc.

It should be understood that RFID technology is described as an example, and that other technologies may also be employed instead of or in addition to RFID technology. The technology employed in the present principles provides features that include an ability of a device to identify its complementary accessory (or device) and to utilize this recognition to allow the device to function. In the absence of the complementary accessory, the device will not function or will be partially disabled and provide warnings or other alert signals. The complementary accessory may be a disposable item, such as, a dressing, but may include other non-disposable devices that may need to be employed together with a complementary device or devices. In one example, a device includes a UV energy source, and the dressing includes an RFID tag built therein to permit the dressing to be recognized by the device. Further, the RFID tag may be used to assist the operator in aligning the device appropriately for delivery of a treatment to the patient. This feature ensures that an effective and safe treatment is delivered to the patient when the accessory is employed, and acts as a point of detection for operators who inadvertently chose the wrong accessory by, e.g., disabling the device.

Patient recognition and record-keeping is another feature. An RFID tag may contain a unique identifier in its stored electronic information, which permits each tag to be differentiated from others. This unique identifier, like a finger print, can be used by the device to determine which patient is being treated. For example, if one device is being used on 50 different patients, the device can discern between patients by reading the RFID tag which is built into the accessory that the patient is wearing (e.g., a disposable dressing). This feature can be used to control whether or not the device will function depending on a treatment schedule, which would mitigate the potential for exceeding the recommended dose in a treatment cycle. For example, a device with a real-time clock and the ability to store data may be able to track the frequency of a patient's treatment, and disable the device function if an appropriate number of hours have not passed since the last dose was administered.

It should be understood that the present invention will be described in terms of medical instruments and accessories; however, the teachings of the present invention are much broader and are applicable to any instrument pairs or sets as well. The present principles are applicable to external or internal procedures of or on biological systems and include procedures in all areas of the body such as the lungs, skin, gastro-intestinal tract, excretory organs, blood vessels, etc. The present principles are applicable to mechanical systems as well where multiple tool pairs or sets are employed. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk read/write (CD-R/W), Blu-Ray™ and DVD.

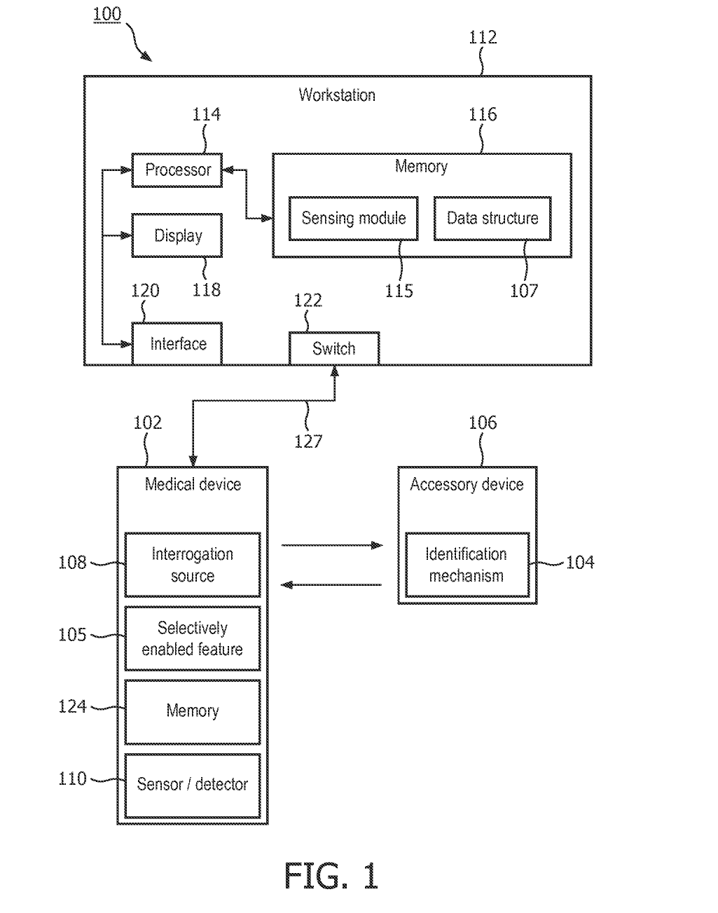

Referring now to the drawings in which like numerals represent the same or similar elements and initially to FIG. 1, a system 100 for detecting compatibility between devices or devices and accessories is illustratively depicted in accordance with one embodiment. System 100 may include a workstation or console 112 from which a procedure is supervised and/or managed. Workstation 112 preferably includes one or more processors 114 and memory 116 for storing programs and applications. Memory 116 may store a sensing module 115 configured to interpret feedback signals from a sensor, detector or mechanism 110 receiving feedback from an identification mechanism 104 of an accessory device 106. In other embodiments, the sensing module 115 may be included on the device 102.

An interrogation source 108 is configured to excite or interrogate the identification mechanism 104 to generate feedback to identify a type of device being used and to compare the feedback against a data structure 107, such as a look-up table or other storage structure stored in memory 116. The sensing module 115 may be included in a device 102 or may receive signals from the medical device 102 as input. The device 102 may include a medical device, although other devices may be employed. In the present example, the medical device 102 may include a catheter, a guidewire, a probe, an endoscope, a robot, an electrode, a filter device, a balloon device, a disinfection handpiece or other medical component. The medical device 102 includes the interrogation source or device 108, which may be configured to wirelessly transmit a signal to the identification mechanism 104 to generate feedback or may include physical attributes for docking or connecting with the sensor device 104 (e.g., contact switches, etc.).

The identification mechanism 104 may include, e.g., an RFID tag, an inductance coil or coils, a bar code, a color generation or photoluminescent material, custom contact switches, a digital image, an image pattern, etc. The identification mechanism 104 is preferably incorporated into the accessory device 106 or other medical device or devices. For example, the identification mechanism 104 may be included in a bandage or dressing material employed as the accessory device 106.

The medical device 102 connects to the workstation 112 through cabling 127. The cabling 127 may include fiber optics, electrical connections, wireless communication, other instrumentation, etc., as needed. The medical device 102 includes the interrogation source 108 and is powered through the workstation 112. The interrogation source 108 provides signals, pulses, radiation or passively measures radiation from the sensor device 104. The medical device 102 is moved in proximity to or aimed at the sensor device 104. The interrogation device 108 is activated to send information, pulses or radiation, etc. to the identification mechanism 104. The sensor device 104 may respond or provide feedback. If no feedback is provided or the incorrect or incompatible feedback is provided, the medical device 102 is partially or fully disabled to prevent operation or the administering of treatment by the device 102.

The feedback from the identification mechanism 104 is input to the workstation 112 and converted or otherwise processed as needed to permit a comparison against the look-up table 107. The look-up table 107 will include any and all compatible signals that will permit enablement of the medical device 102. If the feedback signal is not recognized the medical device 102 is disabled fully or partially. Warning messages may be triggered to let the user know that a compatibility failure has occurred.

The medical device 102 includes a selectively enabled feature or features 105 that are disabled with an incompatibility result determined in the sensing module 115. The medical device 102 may be disabled in a plurality of ways. In one example, the medical device 102 or workstation 112 includes a switch 122, which may be a mechanical/electromechanical switch, a transistor or other integrated circuit switch or a software enabled switch. The switch 122 is closed when a compatibility match occurs and is opened if no compatibility is found between the medical device 102 and the identification mechanism 104. Other methods for disabling the medical device 102 may include locking a trigger, shutting off power, providing a warning, etc.

The medical device 102 may include its own memory 124 or may employ the memory 116 for storing properties or parameters associated with the use of the selectively enabled feature 105. In one embodiment, the selectively enabled feature 105 includes a UV source for disinfecting a dressing or bandage site (accessory device 106).

In one embodiment, workstation 112 includes a display 118 for generating warnings or for other functions needed during a procedure. Display 118 may also permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112. While workstation 112 is depicted with a plurality of features some or all of these features may not be employed, and the workstation 112 still functions in accordance with the present principles. Further, some or all of the features or structures described in the workstation 112 may be implemented in or on the medical device 102 including the processor 114, sensing module 115, switch 122, etc.

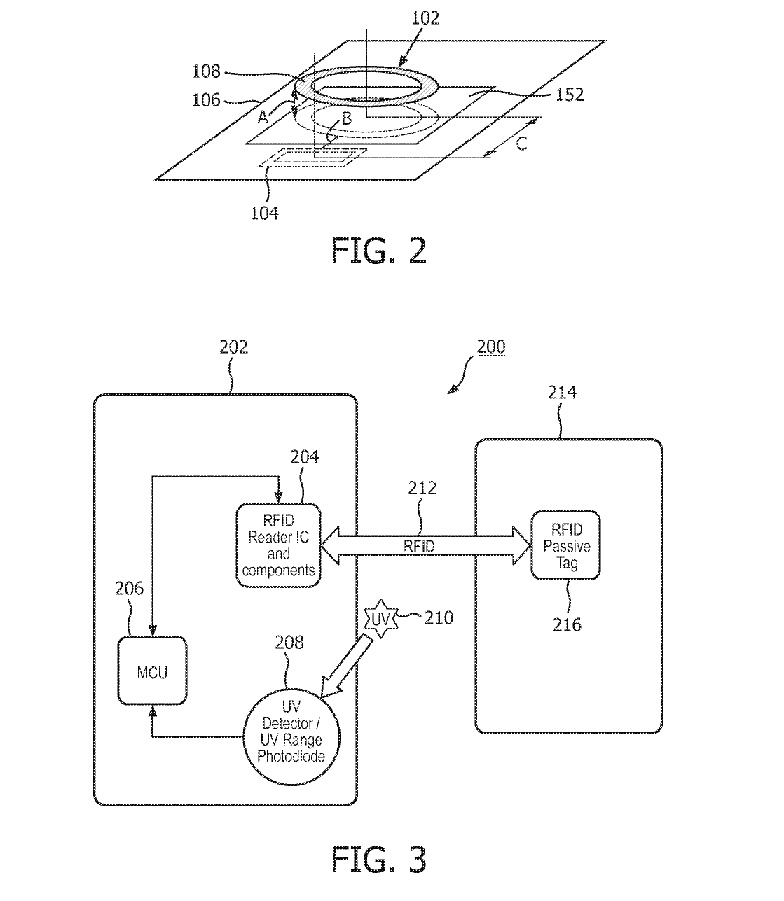

Referring to FIG. 2, a schematic diagram illustratively depicts the interrogation source 108 placed in proximity of the sensor device or identification mechanism 104 on an accessory device 106. The medical device 102 may be brought into close proximity of, e.g., an RFID tag employed as the sensor device 104. The distances “A”, “B” and “C” may be specified and controlled to permit proper reading of the RFID tag, the bar code, etc. A shadow or projection 152 is provided to indicate offsets “B” and “C”. The projection 152 indicates an illustrative treatment area treated by, e.g., a UV light source (not shown).

In one embodiment, the RFID tag 104 is shaped or sized to mark a target or treatment area. The placement, size and shape of the tag 104 forces an operator to align the device (handpiece) 102 and the interrogation source 108 with the RFID tag 104, to ensure treatment is only delivered within that area on the dressing. In other embodiments, multiple RFID tags 104 may be employed on the accessory device 106, as an alternate way of providing alignment guides for an operator to target the center (or other location) of the dressing for delivering treatment.

Referring to FIG. 3, a schematic diagram shows a useful configuration 200 for employing the present principles. Configuration 200 may be implemented with the workstation 112 as described above or may be implemented independently. A UV energy source device or handpiece 202 includes an RFID reader integrated circuit 204, capable of reading an RFID tag 216 within a specified range and orientation. A complementary accessory 214 may include a disposable dressing (bandage) which may include a passive, commercially available RFID tag 216 placed anywhere in the complementary accessory 214. The RFID tag 216 may be permanently affixed to the bandage 214 or may be removable (e.g., semi-permanent) and reused. The device 202 is brought within proximity of the dressing (bandage) 214 such that the reader can identify the RFID tag 216. In this example, a processor or microcontroller unit (MCU) 206 compares feedback received through RF communications 212 to check the compatibility between the device 202 and the accessory 214 using the RFID tag 216.

The RFID reader 204 acknowledges compatibility to the processor 206, which enables functioning of a UV source 210, if compatibility exists. The source 210 may then be used to treat the patient.

RF communications 212 may be low power and short range. The RF range may be measured to provide a “flash safe” distance measurement for the UV source 210. For example, a distance between the source 210 and a patient (accessory 214) can be measured using the RF communications 212. This distance serves to provide an indicator that an effective dose of light has been delivered to a target. A UV detector 208, such as a UV range photodiode may be provided to measure the reflected UV light or measure the intensity or duration of the flash during operation. Such measurements may also be employed to enable or disable functions of the device 202.

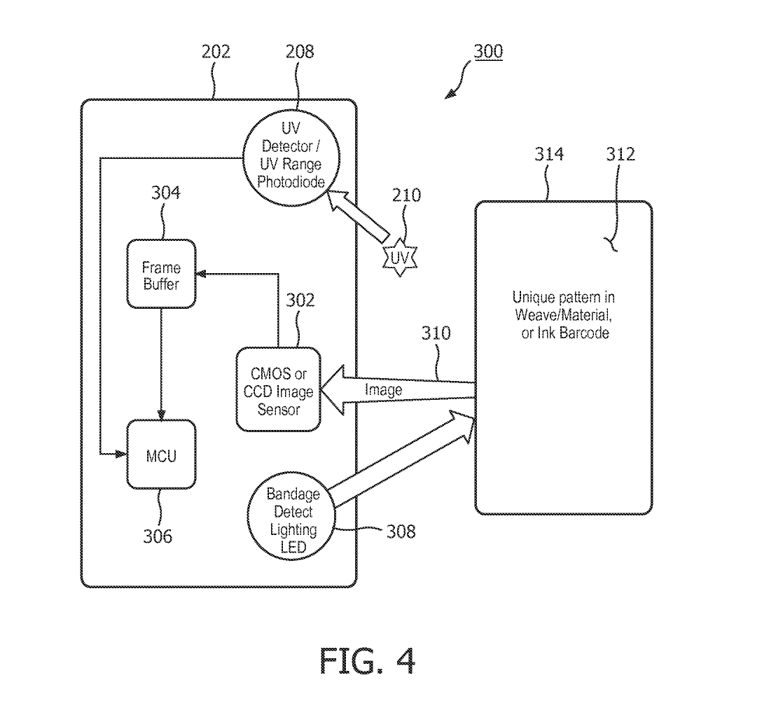

Referring to FIG. 4, a schematic diagram shows another useful configuration 300 for employing the present principles. Configuration 300 may be implemented with the workstation 112 as described above or may be implemented independently. Configuration 300 includes a UV energy source device or handpiece 202 with an image sensor or imager 302, which may include a CMOS or CCD sensing chip or device. A detection light 308 generates light that is directed toward an accessory 314, such as a bandage or dressing. The light 308 may include a light emitting diode or other source configured to read information from the accessory 314. The accessory 314 includes a unique pattern, image or bar code 312 on its surface. The pattern 312 may include an inked pattern, may be woven into fabric of a bandage or dressing, may be a tag or sticker permanently affixed to the surface of the accessory 314, etc.

Reflected light from light source 308 is received in the imager 302. Focus of the image and adjustments made to do so may be employed as a way of determining a distance using focal positioning of a lens of the imager 302. The distance measurement may be employed as a way to ensure correct distance for flash safety, as described above. An image 310 received by the imager 302 is compared to a frame buffer 304 or a plurality of frame buffers, which store compatible images of the pattern 312. If a match exists between the image of the pattern 312 and the images in the frame buffer(s) 304, then the UV source 210 is enabled. The comparison is carried out by a processor (MCU) 306. The processor 306 may also be employed to enable or disable the UV source 210. UV detector 208 may be provided to measure the reflected UV light, measure the intensity or duration of the flash during operation or record a time of treatment. Further, the imager 302 may be employed to detect the flash of the UV source 210 (e.g., its duration, intensity, etc.) by detecting “washout” in the image.

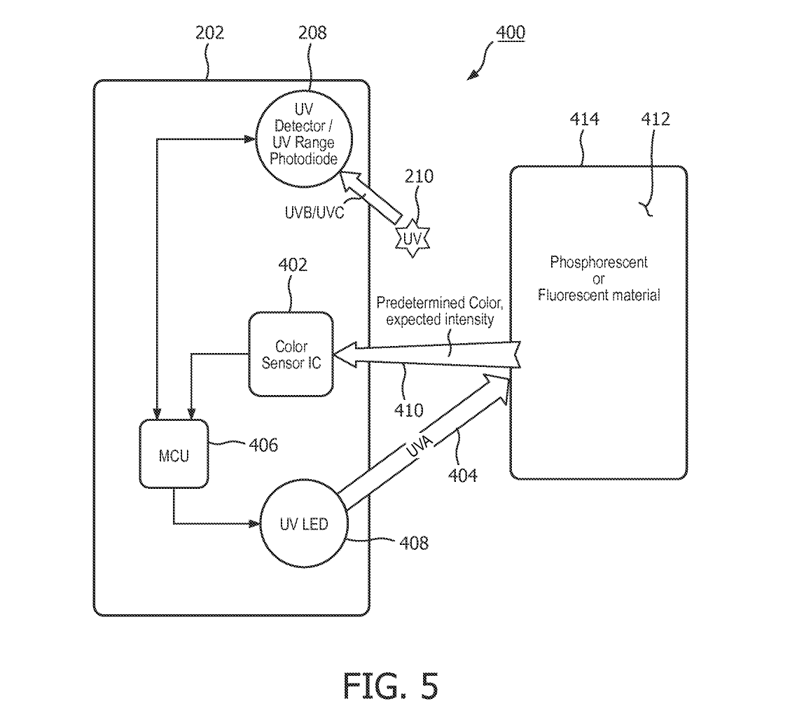

Referring to FIG. 5, a schematic diagram shows another useful configuration 400 for employing the present principles. Configuration 400 may be implemented with the workstation 112 as described above or may be implemented independently. Configuration 400 includes a UV energy source device or handpiece 202 with a color sensor integrated circuit (IC) 402, which may include a CMOS device or the like configured to measure or detect colors. A detection light 408 generates UV light (e.g., UVA as opposed to UVB or UVC generated by UV source 210) that is directed toward an accessory 414, such as a bandage or dressing. The light 408 may include a light emitting diode or other UV source configured to illuminate the accessory 414. The accessory 414 includes a luminescent material 412, such phosphorescent and/or fluorescent material, e.g., lycopodium, calcium sulfide, photoluminescent pigment, etc. These materials may form a pattern or may be uniformly distributed over the accessory 414. Photoluminescent material is not significantly impacted by visible light, but when illuminated with UVA light from the light 408, distinct colors 410 are emitted. The colors 410 may be set to produce a particular wavelength or combinations of wavelengths of light. In addition, the intensity or other properties of the colored light 410 may be employed for comparison to predetermined light properties stored in the color sensor IC 402.

Colored light 410 is received in the IC 402. If the properties are compatible with predetermined settings, the UV source 210 will be enabled. Photoluminescent intensity may also be employed during or after the flash of UV source 210 to determine when the flash occurred and properties about the flash.

If a match exists between the color combinations, then the UV source 210 is enabled. The comparison is carried out by a processor (MCU) 406 and/or the IC 402. The processor 406 may also be employed to enable or disable the UV source 210. UV detector 208 may be provided to measure the reflected UV light or measure the intensity or duration of the flash during operation.

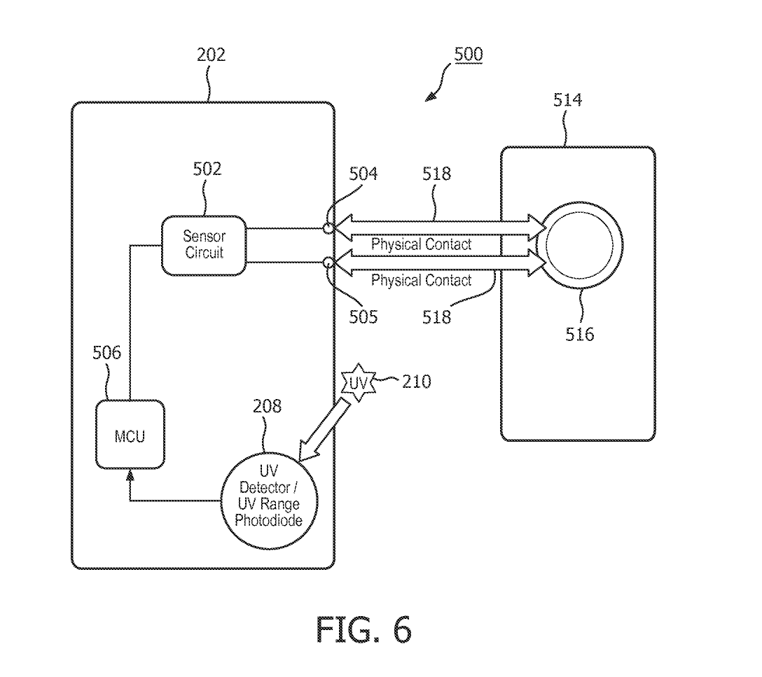

Referring to FIG. 6, a schematic diagram shows another useful configuration 500 for employing the present principles. Configuration 500 may be implemented with the workstation 112 as described above or may be implemented independently. Configuration 500 includes a UV energy source device or handpiece 202 with a sensor circuit 502, which may include a simple circuit configured to provide a voltage or current to/from mechanical contacts 504 and 505. The mechanical contacts 504 and 505 may act as probes to make a measurement of an electrical property from an accessory 514. The accessory 514 includes an exposed conductive shape or circuit 516.

There are several ways in which configuration 500 can be employed. In one embodiment, the contacts 504 and 505 are configured to fit into or against an acceptable or compatible version of the accessory 514 through physical contact 518. If a proper fit is not made than the UV source 210 is disabled by the circuit 502 and/or a processor (MCU) 506. In another embodiment, a physical measurement is made across the contacts 504 and 505. If the measurement is within specifications the accessory 514 is compatible, and the UV source is enabled. The measurement may include, e.g., a resistance value, a capacitance value, an inductance value, combinations of these or others.

Contacts 504, 505 and shape 516 may be made in a plurality of different configurations. In some instances it may be preferable to employ a conductive material for the shape 516 that is compatible with magnetic resonance imaging or other imaging modes. Several configurations of shape 516 may be made compatible with the contacts 504, 505 so that the device 202 can be employed with a plurality of different accessories 514. The shape 516 may include a contact and be made flat or otherwise shaped to provide consistent contact points for contacts 504, 505. The physical contact 518 relationship between contacts 504/505 and 516 may be employed to provide a functional and safe distance between the UV source 210 and the accessory 514 (e.g., which may be worn on a patient). When any one physical contact 518 is broken, the UV source 210 is disabled. It should be noted that the number of contacts may be greater than two, and the number of contacts may be employed in identifying the accessory 514 as well. For example, a shape of concentric rings may be employed where contacts provide connections between the rings at particular locations to measure physical properties, etc. The contacts 504, 505 and shape 516 may have different sizes and extend from their respective surfaces to provide particular relationships therebetween. UV detector 208 may be provided to measure the reflected UV light or measure the intensity or duration of the flash during operation.

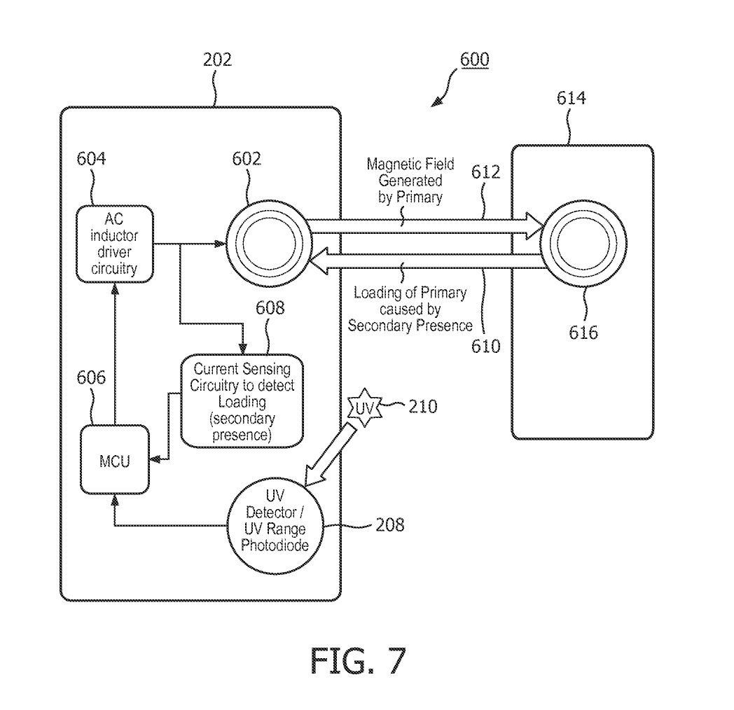

Referring to FIG. 7, a schematic diagram shows another useful configuration 600 for employing the present principles. Configuration 600 may be implemented with the workstation 112 as described above or may be implemented independently. Configuration 600 includes a UV energy source device or handpiece 202 with a primary inductive coil 602 and an AC driver circuit 604 for the inductive coil 602. The driver circuit 604 drives the primary inductive coil 602 to induce a magnetic field 612 in a secondary inductive coil 616 located on an accessory 614 (such as a bandage or the like). Excitation of the secondary coil 616 induces a loading 610 on the primary coil 602 which is measured in a current sensing circuit 608. The sensing circuit 608 detects the presence of a compatible accessory if the sensed parameters meet with expected criteria as determined by a processor (MCU) 606.

The configuration 600 functions as a simplified inductive proximity sensor, which, in turn, permits measurement or control of the distance between the handpiece 202 and the accessory 614. The secondary coil 616 may include a non-ferrous conductor to permit magnetic resonance imaging or other processes. The non-ferrous conductor may include, e.g., NiTi wire, conductive ink, etc. If a proper measurement is not obtained between the inductive coils 602 and 616, then the UV source 210 is disabled by the circuit 608 and/or the processor 606. UV detector 208 may be provided to measure the reflected UV light or measure the intensity or duration of the flash during operation.

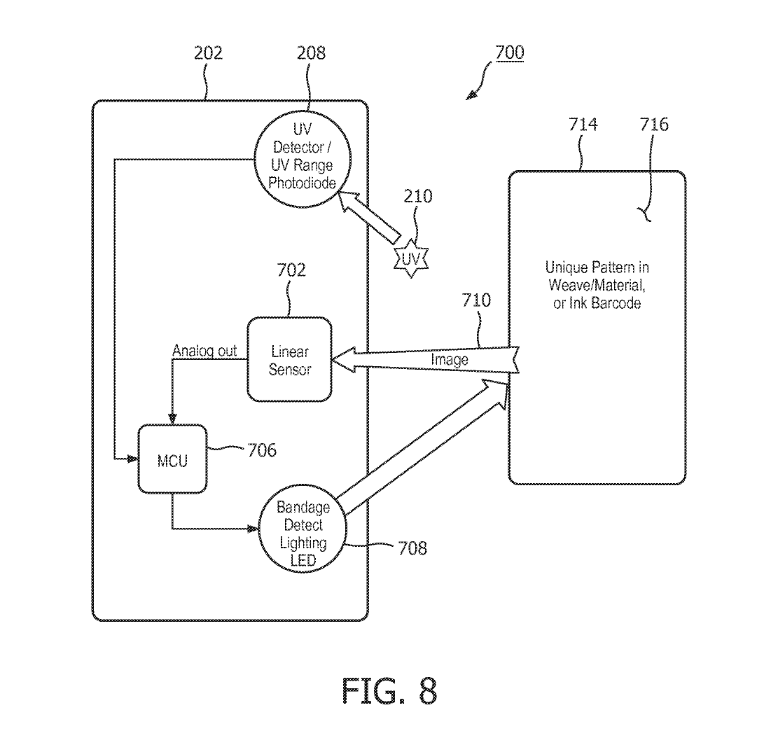

Referring to FIG. 8, a schematic diagram shows another useful configuration 700 for employing the present principles. Configuration 700 may be implemented with the workstation 112 as described above or may be implemented independently. Configuration 700 includes a UV energy source device or handpiece 202 with a linear sensor array 702, which may include a CMOS sensing array or chip. A detection light 708 generates light that is directed toward an accessory 714, such as a bandage or dressing. The light 708 may include a light emitting diode or other source configured to read information from the accessory 714. The light 708 may include visible light. The accessory 714 includes a unique pattern, image or bar code 716 on its surface. The pattern 716 may include an inked pattern, may be woven into fabric of a bandage or dressing, may be a tag or sticker permanently affixed to the surface of the accessory 714.

Reflected light from light 708 is received in the linear sensor array 702. Focus of the image and adjustments made to do so may be employed as a way of determining a distance between the linear sensor array 702 and the accessory 714. The distance measurement may be employed as a way to ensure correct distance for flash safety. An image 704 received by the linear sensor array 702 is compared by the linear sensor array 702 to determine whether a correct or compatible pattern is sensed. If a match exists between the image 704 and the configuration of the linear sensor array 702, an output (e.g., analog) is sent to a processor (MCU) 706 to enable the UV source 210. The comparison is carried out by the linear sensor array 702. The processor 706 is employed to enable or disable the UV source 210 in accordance with the analog output of the linear sensor array 702. UV detector 208 may be provided to measure the reflected UV light or measure the intensity or duration of the flash during operation. Further, the linear sensor array 702 may be employed to detect the flash of the UV source 210 (e.g., its duration, intensity, etc.) by detecting “washout” in the image.

While the present principles have been described in terms of a UV disinfecting system with a bandage or dressing accessory, the present principles are applicable in many other areas of the medical arts as well as in other scientific and engineering pursuits. For example, the present principles may be applied in medical applications, where frequency of dose or treatment is needed for patient health and/or safety or may be applied in a laboratory where dangerous or expensive equipment can be enabled only when a proper accessory or device is employed.

The sensing mechanisms described herein may also be employed for proper positioning of a device during treatment or use for any application where alignment between the device and patient is a consideration. This may also be applied as a tool to prevent incorrect site surgeries, by limiting device function to within the intended surgical site. For example, during dermatological treatments (e.g., tattoo removal) where only a specific site should be treated by the device, an accessory may be placed at the proper location and enablement of the device may be limited to use at that location. In addition, the handpiece 202 in any of the embodiments may include memory storage (e.g., memory 116 and/or 124, FIG. 1). Parameters measured, types, durations, frequencies of treatment, etc. may be stored and associated with unique identifiers on the accessory devices so that records are provided without the need for manual recording.

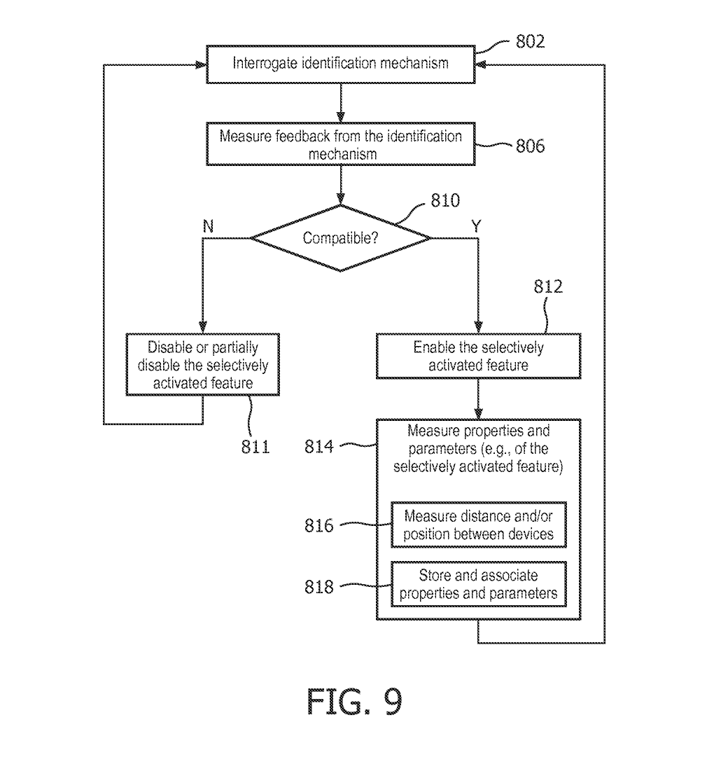

Referring to FIG. 9, a method for determining compatibility to enable device use is shown in accordance with the present principles. In block 802, an identification mechanism of an accessory device is interrogated using an interrogation source to generate feedback from the identification mechanism. The accessory device may include a dressing, bandage or other device. In block 806, the feedback is measured to determine compatibility between a first device (having a selectively activated feature) and the accessory device.

In block 810, a check for compatibility is made. If the feedback indicates compatibility, the selectively activated feature is enabled in block 812. Otherwise, the first device is disabled or partially disabled until compatibility can be established in block 811.

The first device may include a UV disinfection device and the selectively activated feature may include a UV source. In block 814, during enablement of the UV source properties of a flash of the UV source may be measured using a sensor device (detector, etc.). The properties may include intensity, duration, or any other parameter. Other properties may include frequency of use, total exposure time, etc. In block 816, in one embodiment, distance or position of the first device relative to the accessory device may be measured using a relationship between the interrogation device and the identification mechanism. For example, the inductive coils may be employed as position sensors, etc.

In block 818, the properties or parameters associated with the selectively activated feature may be stored in memory and associated with the unique identifiers of the identification mechanism. This feature may obviate the need for manual recording and recordkeeping.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for device sensing in medical applications (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. An accessory enabled device, comprising:
a first device including a selectively activated feature;
said first device including an interrogation source configured to interrogate an accessory device, such that, responsive to an interrogation, feedback from the accessory device is measured to determine compatibility between the first device and the accessory device;
the selectively activated feature being enabled to perform a task only when a specified compatibility exists between the first device and the accessory device; and
the device is configured to determine that the specified compatibility exists only when the first device and the accessory device have a predetermined spatial relationship with respect to each other, said spatial relationship ensuring proper alignment of the accessory enabled device for a procedure to be performed by the accessory enabled device.

2. The device as recited in claim 1, wherein the first device includes an ultra-violet (UV) disinfection device and the selectively activated feature includes a UV source.

3. The device as recited in claim 2, wherein the first device includes a UV detector to measure properties of a flash of the UV source.

4. The device as recited in claim 1, wherein the accessory device includes a dressing or bandage.

5. The device as recited in claim 1, wherein the interrogation source includes a radio frequency identification (RFID) reader and the accessory device includes an RFID tag.

6. The device as recited in claim 1, wherein the interrogation source includes a light emitter and the accessory device includes a discernable pattern.

7. The device as recited in claim 6, wherein the discernable pattern includes one of a bar code, an inked pattern and/or a surface texture.

8. The device as recited in claim 6, wherein the light emitter includes visible light and the discernable pattern is visible.

9. The device as recited in claim 1, wherein the interrogation source includes an ultra-violet light emitter and the accessory device includes a photoluminescent material.

10. The device as recited in claim 1, wherein the interrogation source includes a first contact configuration and the accessory device includes a second contact configuration.

11. The device as recited in claim 1, wherein the interrogation source includes a primary coil configuration and the accessory device includes a secondary coil configuration.

12. The device as recited in claim 1, wherein the feedback from the accessory device is employed to measure one of a distance or position of the first device relative to the accessory device.

13. The device as recited in claim 1, further comprising a memory device configured to store parameters associated with the selectively activated feature.

14. A system for determining compatibility between devices, comprising:
a first device including a selectively activated feature;
a second device including an identification mechanism configured to identify a characteristic of the second device;
said first device including an interrogation source configured to interrogate the second device, such that, responsive to an interrogation, feedback from the identification mechanism is measured to determine compatibility between the first device and the second device;
the selectively activated feature being enabled to perform a task only when compatibility exists between the first device and the second device; and
the system is configured to determine that compatibility exists between the first device and the second device only when there is a predetermined spatial relationship between the first device and second device, said spatial relationship ensuring proper alignment of the first device and second device for a procedure to be performed.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. A method for determining compatibility to enable device use,
comprising:
interrogating an identification mechanism of an accessory device using an interrogation source to generate feedback from the identification mechanism;
measuring the feedback to determine compatibility between a first device having a selectively activated feature and the accessory device; and
if the feedback signal indicates compatibility, enabling the selectively activated feature, wherein the first device and the accessory device must have a predetermined spatial relationship with respect to each other in order for the feedback signal to indicate compatibility, said spatial relationship ensuring proper alignment of the first device and the accessory device for a procedure to be performed.

27. (canceled)

28. (canceled)

29. (canceled)

30. (canceled)

31. (canceled)

32. (canceled)
